(19) United States
(12) Patent Application Publication
(10) Pub. No.: US 2010/0228319 A1
(43) Pub. Date: Sep. 9, 2010

(54) ELECTRICAL STIMULATION SYSTEM AND METHOD USING MULTI-GROUP ELECTRODE ARRAY

(75) Inventors: CHARLES TAK-MING CHOI, Hsinchu City (TW); CHIEN-HUA HSU, Hsinchu County (TW)

Correspondence Address: WPAT, PC;INTELLECTUAL PROPERTY ATTORNEYS, 2030 MAIN STREET, SUITE 1300, IRVINE, CA, 92614, US

(73) Assignee: NATIONAL CHIAO TUNG UNIVERSITY, HSINCHU CITY (TW)

(21) Appl. No.: 12/719,022

(22) Filed: Mar. 8, 2010

(30) Foreign Application Priority Data
Mar. 9, 2009 (TW) 098107586

(51) Int. Cl. A61N 1/05

(52) U.S. Cl. 607/45; 607/57

(57) ABSTRACT

An electrical stimulation system and method using multi-group electrode array are disclosed. The electrical stimulation system comprises an implant body having an electrode carrying surface, an electrode array provided on the electrode carrying surface, and an electrode controller. The electrode array comprises a plurality of electrode groups, each of which includes a plurality of electrodes, and each of the electrodes has an independent power supply control. The electrode controller receives a control signal to drive a set of at least two corresponding electrodes, which is selected from the electrodes of the same electrode group and two adjacent electrode groups. Powers supplies to the set of corresponding electrodes may be regulated by the independent power supply controls thereof, so as to generate a virtual channel between the set of corresponding electrodes through interaction of powers supplied to the set of corresponding electrodes.

### FIELD OF THE INVENTION

The present invention relates to an electrical stimulation system and method using multi-group electrode array, and more particularly, to an electrical stimulation system that uses a multi-group electrode array to control the target stimulation sites and directions for sending stimulating powers to nerve fibers.

### BACKGROUND OF THE INVENTION

An electrical stimulation system is a system capable of receiving a control signal containing a control parameter, generating stimulating currents corresponding to the control parameter, and sending the stimulating currents to disabled or malfunctioning organs or body areas to produce an instant effect to replace or correct the disabled or malfunctioning organs or body areas. Currently, electrical stimulation systems are mainly applied in cochlear implants (CI) or deep brain stimulations (DBS). A cochlear implant system first captures speeches or sounds through a microphone. Next, a speech processor will convert the sound wave into signal in different audio frequency, and then, an electrode corresponding to the audio frequency generates a stimulating current, which is fed to a patient's cochlear to stimulate the corresponding auditory nerves to compensate for the patient's hearing function. When the electrical stimulation system is applied in a DBS system, electrodes are implanted in the patient's brain close to the target area, abnormal tissues, to generate stimulating currents, so as to suppress the symptoms caused by abnormal electric discharge of the abnormal tissues.

FIGS. 1 and 2 are schematic perspective and plan views, respectively, showing the structure of a conventional electrical stimulation system 1. As shown, the electrical stimulation system 1 comprises an implant body 11, a plurality of electrodes 12, and an electrode controller 13. The implant body 11 has an electrode carrying surface, on which an electrode array 14 comprising the electrodes 12 is provided. The electrodes 12 have a common power supply control. The electrode array 14 is electrically connected to the electrode controller 13, which supplies electric currents to the electrodes, and drives the electrodes 12 when receiving a control signal containing a control parameter, such as an audio frequency. The electric currents supplied to the electrodes 12 is regulated by the power supply control, and other parameters, such as the current intensity and current ratio, are changed according to the control parameters, so that the electrodes 12 generate stimulating currents corresponding to the control parameters.

In the conventional electrical stimulation system 1, the stimulating currents are generated by the electrodes 12, and the positions of the electrodes 12 in the electrode array 14 are not arbitrarily changeable once the electrode array 14 has been implanted in the patient's body. Therefore, the positions of the electrodes 12 in the electrode array 14 will form a limit to the stimulation directions of the stimulating currents. When the electrodes 12 are ring electrodes, the stimulating currents generated by the electrodes 12 produce isotropic stimulation, as indicated by the arrows 15 in FIG. 1. As a result, when the electrical stimulation system 1 is applied to a patient whose auditory nerve is degenerated at nerve ending and only part of the nerve cells or axons are remained, there are chances of having a poor stimulation effect due to improper stimulation directions of the stimulating currents. Or, when the electrical stimulation system 1 is applied to a patient with Parkinson's disease, any deviation of the electrodes 12 in position will very possibly cause poor stimulation effect. The position-deviated electrodes must be re-implanted via another implantation surgery and tend to undesirably stimulate other tissues and cause abnormal side effects. The above-mentioned undesirable conditions might cause worse results when the stimulating currents produce isotropic stimulation. Therefore, it appears that the conventional electrical stimulation system 1 is not necessarily suitable for all patients because the target stimulation sites and stimulation directions of the stimulating currents may not be finely adjusted according to different patients' conditions and symptoms.

The aforementioned drawbacks in the conventional electrical stimulation system can be corrected through the virtual channel technique. The virtual channel technique is based on a technique called current steering, in which two adjacent electrodes 12 in the electrode array 14 are simultaneously driven for them to generate stimulating currents at the same time. It is known that two stimulating currents generated at the same time will interact with each other. Therefore, by controlling the phases of the two stimulating currents and the ratio thereof, an integrated stimulating current between the two physical electrodes can be generated to stimulate the nerve tissues, such as the auditory nerves of a cochlear implantee. The stimulation so produced is located between two physical channels produced by individually driving two physical electrodes, and therefore an additional channel may be perceived. Such an additional channel is referred to as a virtual channel or a virtual electrode. Further, by controlling the virtual channel, the target stimulation sites and the stimulation directions may be adjusted without being limited by the physical channels produced by the physical electrodes. However, since the electrodes generate stimulating currents through power control, when the target stimulation sites and the stimulation directions of the virtual channel are controlled simply by applying different current ratio combinations, only a relatively low resolution of the available virtual channels can be obtained due to limited current ratio combinations, and the target stimulation sites and the stimulation directions of the virtual channels may not be easily fine tunable.

It is therefore proposed by the present inventors to develop an electrical stimulation system and method using multi-group electrode array, so as to overcome the drawbacks in the conventional electrical stimulation system.

### SUMMARY OF THE INVENTION

A primary object of the present invention is to provide an electrical stimulation system using multi-group electrode array, so as to more accurately control the target stimulation sites and stimulation directions of virtual channels.

To achieve the above and other objects, the electrical stimulation system using multi-group electrode array according to the present invention includes an implant body having an electrode carrying surface, an electrode array provided on the electrode carrying surface, and an electrode controller. The electrode array comprises a plurality of electrode groups, each of which includes a plurality of electrodes having an independent power supply control each. The electrode controller receives a control signal to drive a set of at least two corresponding electrodes. Power supply to the set of corresponding electrodes can be regulated by the corresponding independent power supply controls thereof, so that a virtual channel is generated between the corresponding electrodes through interaction of the powers supplied to the set of corresponding electrodes.

Each of the set of at least two corresponding electrodes is selected from the group consisting of the electrodes of the same electrode group and the electrodes of two adjacent electrode groups.

The control signal comprises a control parameter, and the control parameter is an output ratio of powers.

The electrical stimulation system further comprises a mapping table, from which a corresponding relationship between the control parameter and virtual channel is able to be found. And, the mapping table may be created by using any one of the genetic algorithm, the ant algorithm, or other optimization algorithms.

The electrodes in each of the electrode groups forming the electrode array may be arranged into different patterns, such as a triangular pattern, a rectangular pattern, a pentagonal pattern, a hexagonal pattern, or a partial circular pattern.

The electrodes are select from the group consisting of a monopolar form, a bipolar form, and a multi-polar form.

The powers supplied to the set of corresponding electrodes is selected from the group consisting of a voltage source and a current source.

The electrical stimulation system using multi-group electrode array is applicable to cochlear implant or deep brain stimulation.

The electrical stimulation method using multi-group electrode array according to the present invention includes the following steps: providing an implant body having an electrode carrying surface; providing an electrode array on the electrode carrying surface of the implant body, the electrode array comprising a plurality of electrode groups, each of which including a plurality of electrodes, and the electrodes each having an independent power supply control; receiving a control signal via an electrode controller to drive a set of at least two corresponding electrodes; and regulating the powers supplied to the set of corresponding electrodes by the corresponding independent power supply controls thereof, so as to generate a virtual channel between the set of corresponding electrodes through interaction of powers supplied to the set of corresponding electrodes.

Each of the set of at least two corresponding electrodes may be selected from the group consisting of the electrodes of the same electrode group and the electrodes of two adjacent electrode groups.

The control signal comprises a control parameter, and the control parameter is an output ratio of powers.

In the electrical stimulation method, further comprising a step of using a mapping table to look up the corresponding relationship between the control parameters and the virtual channels. And, the mapping table may be created by using any one of the genetic algorithm, the ant algorithm, or other optimization algorithms.

The electrodes in each of the electrode groups forming the electrode array may be arranged into different patterns, such as a triangular pattern, a rectangular pattern, a pentagonal pattern, a hexagonal pattern, or a partial circular pattern.

The electrodes are selected from the group consisting of a monopolar form, a bipolar form, and a multi-polar form.

The powers supplied to the set of corresponding electrodes is selected from the group consisting of a voltage source and a current source.

The electrical stimulation method using multi-group electrode array can is applicable to cochlear implant or deep brain stimulation.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a schematic perspective view showing the structure of a conventional electrical stimulation system;

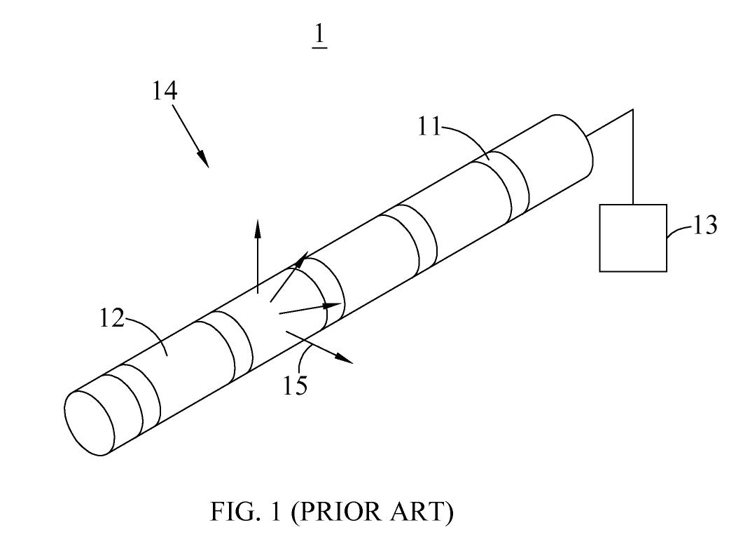

FIG. 2 is a plan view of the conventional electrical stimulation system of FIG. 1;

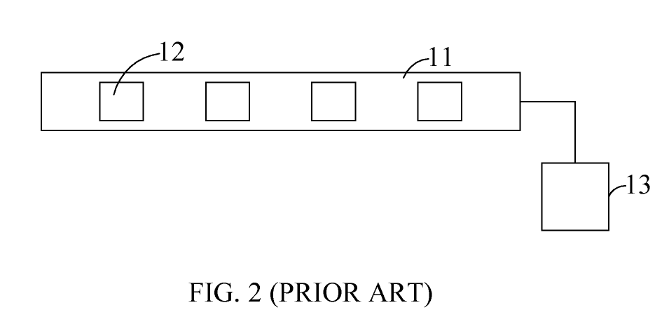

FIG. 3 is a schematic perspective view showing the structure of an electrical stimulation system using multi-group electrode array according to a first preferred embodiment of the present invention;

FIG. 4 is a diagram of activation function curves according to virtual channels generated between different sets of corresponding electrodes when the electrical stimulation system of the present invention is applied in cochlear implant;

FIG. 5 is a schematic view of an electrical stimulation system according to an embodiment of the present invention having a plane electrode carrying surface;

FIG. 6 is a schematic view of an electrical stimulation system according to an embodiment of the present invention having an angular electrode carrying surface;

FIG. 7 is a schematic view of an electrical stimulation system according to an embodiment of the present invention having a spherical electrode carrying surface;

FIG. 8 is a schematic structural view of an electrical stimulation system of the present invention showing the electrodes in each of the electrode groups forming the electrode array may be arranged into different patterns;

FIG. 9 is a perspective view showing the structure of an electrical stimulation system using multi-group electrode array according to a second preferred embodiment of the present invention;

FIG. 10 is a sectioned perspective view of the electrical stimulation system of FIG. 9 showing one of the electrode groups thereof;

FIG. 11 exemplifies some possible top plan views of the electrical stimulation system of FIG. 9 showing different quantities of electrodes can be included in an electrode group; and

FIG. 12 is a flowchart showing the steps included in an electrical stimulation method using multi-group electrode array according to the present invention.

### DETAILED DESCRIPTION OF THE PREFERRED EMBODIMENTS

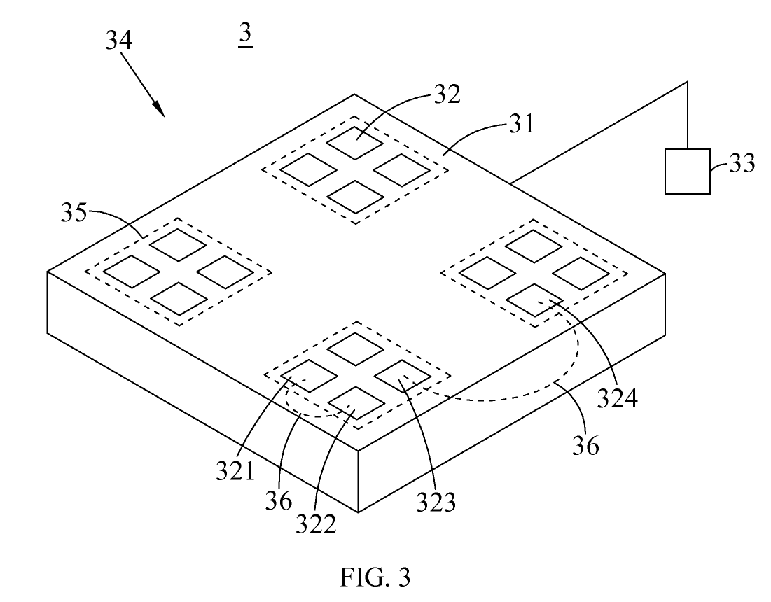

Please refer to FIG. 3 that is a schematic perspective view showing the structure of an electrical stimulation system using multi-group electrode array according to a first preferred embodiment of the present invention. As shown, the electrical stimulation system, which is generally denoted a reference numeral 3, includes an implant body 31, an electrode array 34, and an electrode controller 33. The implant body 31 has an electrode carrying surface, on which the electrode array 34 is provided. The electrode array 34 comprises a plurality of electrode groups 35, and each of the electrode groups 35 includes a plurality of electrodes 32. Moreover, each of the electrodes 32 has an independent power supply control. The electrode array 34 is electrically connected to the electrode controller 33, which drives one set of at least two corresponding electrodes when receiving a control signal containing selection parameters and control parameters for, for example, selecting output ratio of powers supplied to electrodes. Through independent power supply controls, the power supplied to the set of corresponding electrodes may be regulated. The powers supplied to the set of corresponding electrodes may be selected from the group consisting of a voltage source and a current source. Through interaction of currents, a virtual channel may be generated between the set of at least two corresponding electrodes.

Based on the selection parameter, mutually corresponding electrodes (i.e. corresponding electrodes) are selected from the electrodes in the same electrode group 35 and in two adjacent electrode groups 35. For the purpose of easy to describe, in the first preferred embodiment illustrated in FIG. 3, the selected corresponding electrodes include a first electrode 321 and a second electrode 322. Through the independent power supply controls thereof, the powers supplied to the first electrode 321 and the second electrode 322 are regulated. Meanwhile, based on the control parameter, other parameters, such as current intensity and current ratio, are changed to generate a virtual channel 36 between the first and the second electrode 321, 322 corresponding to the control parameter. However, the number of the selected corresponding electrodes is not necessarily limited to two, such as the first and the second electrode 321, 322, but can be more than two. In addition, for the purpose of providing convenience in practical application, a mapping table may be created in advance. From the mapping table, a user may look up the corresponding relationship between the control parameters and different virtual channels. The mapping table may be created by using genetic algorithm, ant algorithm, or other optimization algorithms.

The virtual channel 36 is generated between corresponding electrodes, and the corresponding electrodes may be selected from the electrodes of the same one electrode group 35, such as the above-exemplified first and second electrodes 321, 322, and from the electrodes in two adjacent electrode groups 35, such as a third electrode 323 and a fourth electrode 324 shown in FIG. 3. Therefore, a plurality of virtual channel 36 may be generated at different positions in the same one electrode group 35 or between two adjacent electrode groups 35.

In the electrical stimulation system 3 of the present invention, the positions of corresponding electrodes for generating the virtual channels 36 therebetween may be adjusted by the electrode controller 33. This may be achieved by regulating the powers supplied to the selected corresponding electrodes through the independent power supply controls thereof, so as to change other different parameters, such as current intensity and current ratio.

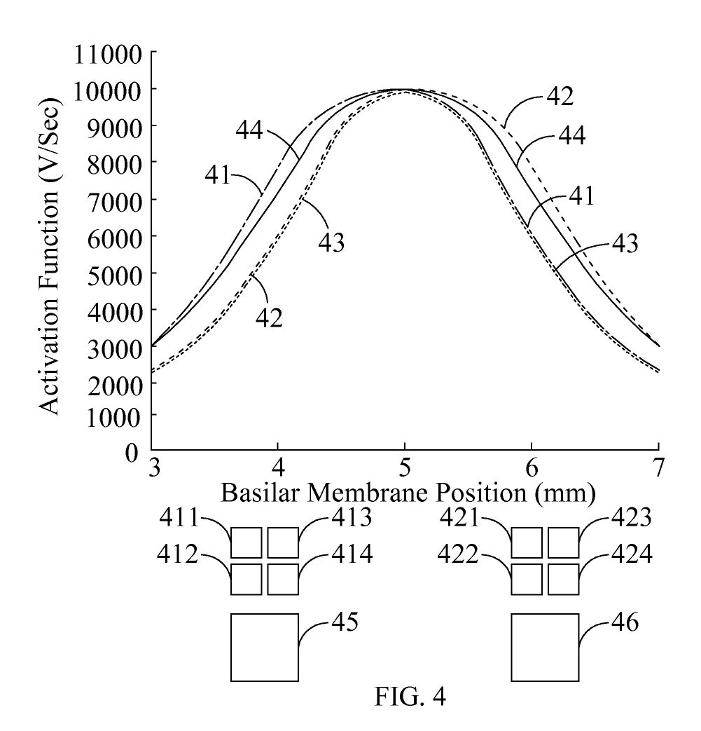

FIG. 4 is a diagram of activation function curves according to virtual channels 36 generated between different sets of corresponding electrodes when the electrical stimulation system 3 of the present invention is applied in cochlear implant. From the activation function curve diagram, it may be seen the stimulation effects achieved when the electrical stimulation system 3 adjusts the positions of the corresponding electrodes for generating virtual channels therebetween. As a reference, since the electrode array 34 implanted in a human ear is disposed along the basilar membrane in the cochlear, the corresponding electrodes are also correspondingly located at the basilar membrane. In the activation function curve diagram of FIG. 4, the horizontal axis indicates different positions along the basilar membrane measured in the unit of millimeter (mm). A plurality of electrodes in two electrode groups are shown below the horizontal axis. The two electrode groups are separately located at the 4 mm and the 6 mm position along the basilar membrane. The activation function indicates the degree of activation for the auditory nerves through the virtual channel 36. A higher activation function value indicates the nerves are more likely to be stimulated. When the intensity of the virtual channel 36 exceeds a threshold value, the auditory nerve corresponding to the control parameter, such as audio frequency, represented by the virtual channel 36 will be excited. Activation of the auditory nerves corresponding to the 4 mm position along the basilar membrane will generate a perception of higher frequency than those at the 6 mm position.

In FIG. 4, the electrode group located at the 4 mm position along the basilar membrane may include a plurality of electrodes, such as electrodes 411-414. Similarly, the electrode group located at the 6 mm position along the basilar membrane may include a plurality of electrodes, such as electrodes 421-424. When the selected corresponding electrodes are electrodes 411 and 421, a virtual channel is generated between the two electrodes 411, 421, and the activation function curve thereof is the curve 41. When the selected corresponding electrodes are electrodes 413 and 423, a virtual channel is generated between the two electrodes 413, 423, and the activation function curve thereof is the curve 42. Or, when the selected corresponding electrodes are electrodes 413 and 421, a virtual channel is generated between the two electrodes 413, 421, and the activation function curve thereof is the curve 43. For the purpose of comparison, two conventional single electrodes 45, 46 are also separately disposed at the 4 mm and the 6 mm position along the basilar membrane, and the activation function curve of the virtual channel generated therebetween is the curve 44. As can be found from FIG. 4, among the four curves 41˜44, the curve 43 has the smallest beam width. This means the virtual channel generated between the two electrodes 413, 421 may produce more focused stimulation than the virtual channels generated at other positions. From the aforementioned description, the peak of the virtual channels can be steered by using hardware, e.g. moving from 411-421 pair to 413-423 pair, or by adjusting the ratio of the magnitudes of the current at 411 and 421.

From the above description, it is understood the electrical stimulation system 3 of the present invention allows adjustment of the positions of the selected sets of corresponding electrodes for generating the virtual channels by controlling different parameters, such as current intensity and current ratio. Therefore, in the electrical stimulation system 3 of the present invention, the virtual channels 36 may be generated between corresponding electrodes located in the same electrode group 35 or in two adjacent electrode groups 35. And, in the case of generating the virtual channel between two electrode groups 35, the positions of the virtual channels may be adjusted through proper planning of electrode groups and selection of corresponding electrodes. Moreover, the positions of corresponding electrodes for generating the virtual channels may also be adjusted by controlling different parameters, such as current intensity and current ratio. Further, the target stimulation sites and the stimulation directions of the virtual channels 36 may be finely adjusted through selection of the positions of the electrode groups and the corresponding electrodes for generating the virtual channels 36, so as to achieve a high stimulation resolution.

Since each of the electrode groups 35 includes a plurality of electrodes 32, and the corresponding electrodes are selected from the electrodes 32 of the same electrode group 35 and two adjacent electrode groups 35, the higher the number of the electrodes 32 is, the higher stimulation resolution may be achieved.

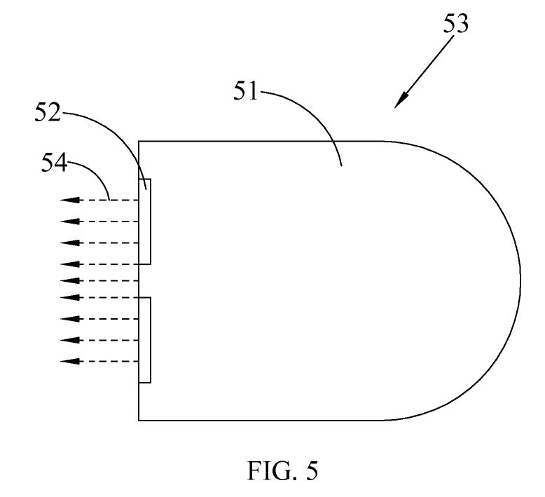

Please refer to FIG. 5 that is a schematic structural view of an electrical stimulation system according to an embodiment of the present invention, of which an implant body 51 has an electrode carrying surface, on which an electrode array 53 is provided. The electrode array 53 comprises a plurality of electrode groups, each of which includes a plurality of electrodes 52. FIG. 5 is a sectional view taken along a plane comprising one of the electrode groups. Since the implant body 51 in this embodiment has a plane electrode carrying surface, the corresponding electrodes 52 selected from the electrode group are also located on a plane surface, and the virtual channels generated between the selected sets of corresponding electrodes 52 have parallel stimulation directions, as indicated by the arrows 54. Therefore, a vertical range of the stimulation directions of the virtual channels can be controlled.

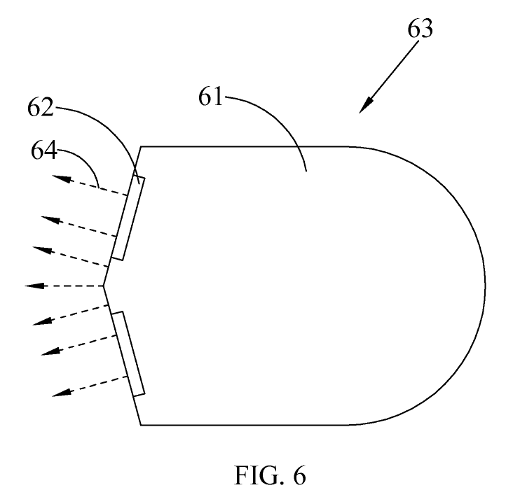

Please refer to FIG. 6 that is a schematic structural view showing an electrical stimulation system according to another embodiment of the present invention, of which an implant body 61 has an electrode carrying surface, on which an electrode array 63 is provided. The electrode array 63 comprises a plurality of electrode groups, each of which includes a plurality of electrodes 62. FIG. 6 is a sectional view taken along a plane containing one of the electrode groups. Since the implant body 61 in this embodiment has an angular electrode carrying surface, the corresponding electrodes selected from the electrode group are also located on an angular surface, and the virtual channels generated between the selected sets of corresponding electrodes have angularly spaced stimulation directions, as indicated by the arrows 64. In this embodiment, the range between the upper and the lower stimulation is larger than that in the embodiment shown in FIG. 5.

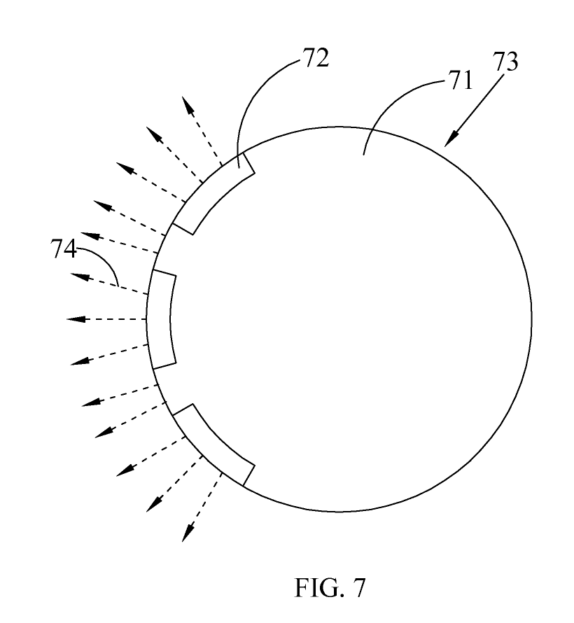

Please refer to FIG. 7 that is a schematic structural view showing an electrical stimulation system according to a further embodiment of the present invention, of which an implant body 71 has an electrode carrying surface, on which an electrode array 73 is provided. The electrode array 73 comprises a plurality of electrode groups, each of which includes a plurality of electrodes 72. FIG. 7 is a sectional view taken along a plane containing one of the electrode groups. Since the implant body 71 in this embodiment has a spherical electrode carrying surface, the corresponding electrodes selected from the electrode group are also located on a spherical surface, and the virtual channels generated between the selected sets of corresponding electrodes have radial stimulation directions, as indicated by the arrows 74.

As can be seen from FIGS. 5, 6 and 7, the virtual channels generated between the corresponding electrodes 72 provided on the spherical electrode carrying surface of the implant body 71 have the broadest range between the upper and the lower stimulation directions, as compared to those in the embodiments shown in FIGS. 5 and 6. Therefore, according to the spirit of the present invention, the electrode carrying surface on the implant body may be determined according to the desired stimulating directions of the virtual channels.

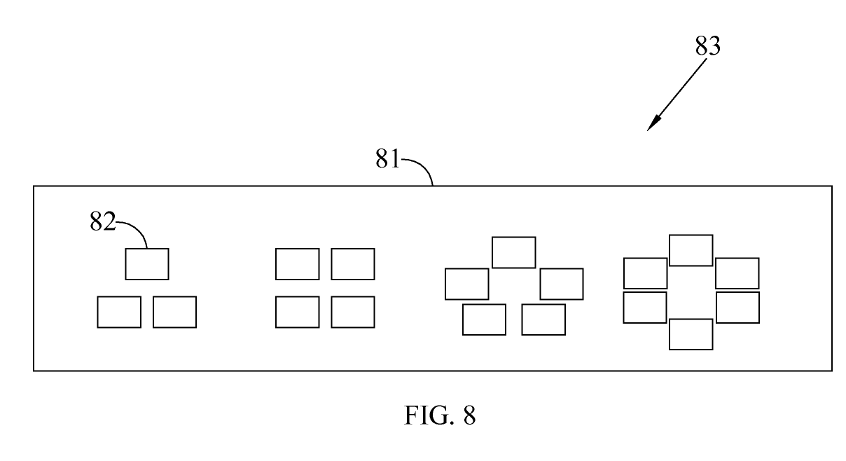

FIG. 8 is a schematic structural view of an electrical stimulation system according to a still further embodiment of the present invention. In this embodiment, the electrical stimulation system includes an implant body 81 and a plurality of electrodes 82. The implant body 81 has an electrode carrying surface, on which an electrode array is provided. The electrode array 83 comprises a plurality of electrode groups, each of which includes a plurality of the electrodes 82. The electrodes 82 in the electrode groups may be arranged into different patterns, such as a triangular pattern, a rectangular pattern, a pentagonal pattern, and a hexagonal pattern as illustrated in FIG. 8, or other patterns, such as a partial circular pattern. With these different arrangements, the virtual channels may have various stimulation directions, such as lateral stimulation directions, vertical stimulation directions, and diagonal stimulation directions. The more electrodes are included in one electrode group, the higher the diversity and flexibility of the stimulating directions available from the virtual channels may be. However, this will also result in higher manufacturing cost. Therefore, according to the spirit of the present invention, the arrangements of the electrodes 82 on the implant body 81 can be adjusted according to the desired positions for generating the virtual channels.

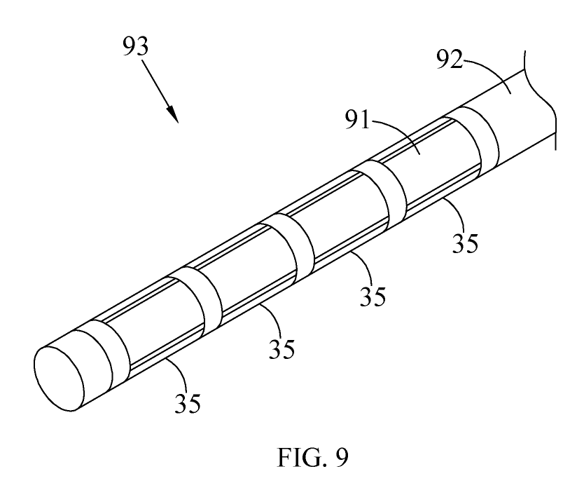

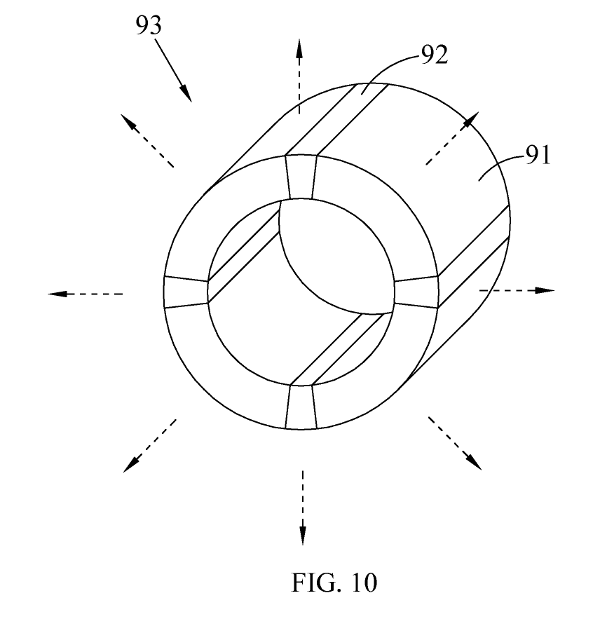

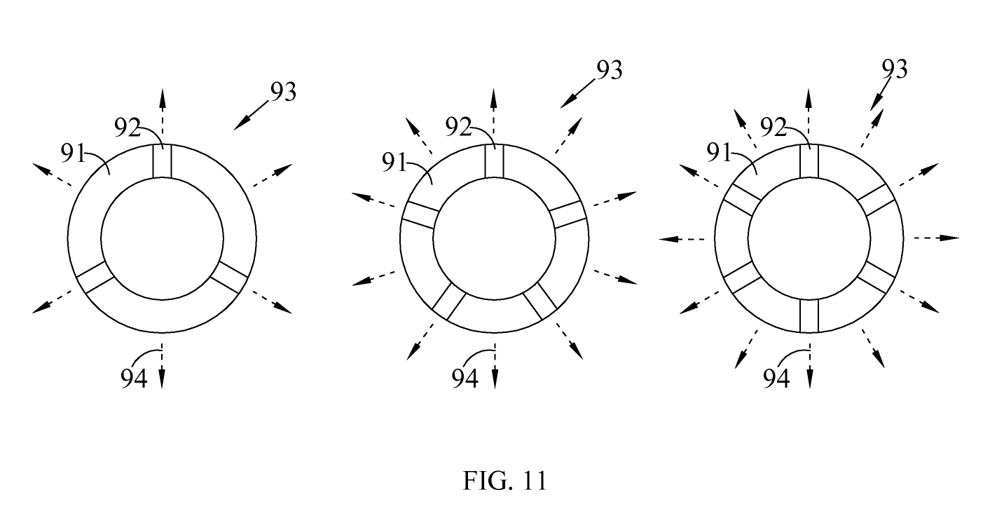

FIGS. 9, 10 and 11 show an electrical stimulation system according to a second preferred embodiment of the present invention. As shown, the electrical stimulation system in this embodiment includes an implant body 92 and a plurality of electrode groups 35, each of which includes a plurality of electrodes 91. The implant body 92 has an electrode carrying surface, on which an electrode array 93 is provided. The electrode array 93 comprises the electrode groups 35, each of which includes a plurality of electrodes 91. As illustrated in FIG. 9, each of the electrode groups 35 is arranged around the implant body 92 to form one circle, and there are four electrode groups 35 on the implant body 92 to form the electrode array 93. FIG. 10 is a fragmentary sectioned perspective view of FIG. 9 showing one of the electrode groups 35 arrayed on the implant body 92. FIG. 11 exemplifies some top plan views showing the electrode groups for the electrical stimulation system of FIG. 9 may have different quantities of electrodes 91 included therein according to actual need. With the electrodes 91 in the electrode groups arranged around the implant body 92, the generated virtual channels 94 may have radial stimulation directions within 360°. The number of electrodes 91 in each electrode group is not particularly limited. FIG. 11 illustrates three different electrode groups with three, five, and six electrodes 91, respectively. Of course, the electrode group may include any other number of electrodes 91. The more electrodes are included in one electrode group, the more accurate angles for the virtual channels may be produced. However, the manufacturing cost will increase with the increased number of electrodes. Therefore, according to the spirit of the present invention, the number of electrodes in the electrode groups can be adjusted according to the desired positions for generating virtual channels.

In the present invention, in addition to the above-mentioned monopolar form (i.e. having a working electrode and a remote reference electrode), the electrodes may be otherwise in a bipolar form or a multi-polar form. In the case of a bipolar form, it means the electrodes comprise a working electrode and a proximate reference electrode. The reference electrode is used as a reference point for describing the potential of the working electrode.

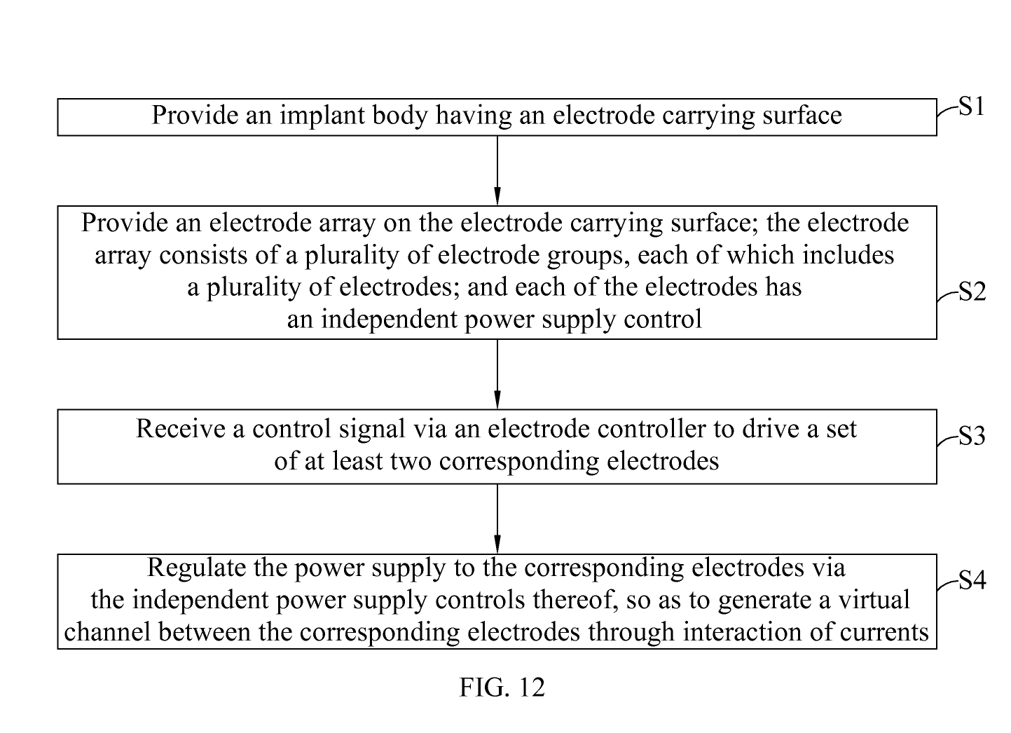

FIG. 12 is a flowchart showing the steps included in an electrical stimulation method using multi-group electrode array according to the present invention. In a first step S1, an implant body having an electrode carrying surface is provided. The electrode carrying surface may be a plane surface, an angular surface, or a spherical surface, as shown in FIGS. 5, 6 and 7, respectively. In a second step S2, an electrode array is provided on the electrode carrying surface of the implant body. The electrode array comprises a plurality of electrode groups, each of which includes a plurality of electrodes, and the electrodes each have an independent power supply control. The electrodes in each electrode group may be arranged into a triangular pattern, a rectangular pattern, a pentagonal pattern, a hexagonal pattern, or a partial circular pattern, as shown in FIG. 8. In a third step S3, a control signal is received via an electrode controller to drive a set of at least two corresponding electrodes. The corresponding electrodes in the same set may be selected from the electrodes included of the same one electrode group and two adjacent electrode groups. And, in a fourth step S4, adjust the powers supplied to the set of corresponding electrodes via the independent power supply controls thereof, so as to generate a virtual channel between the set of corresponding electrodes via interaction of powers. An output ratio of powers for the corresponding electrodes may be controlled via a control signal comprising a control parameter. The power supply may be a voltage source or a current source. In addition, a mapping table may be utilized to look up the corresponding relationship between the control parameter and different virtual channels. In this manner, a user may conveniently control the target stimulation sites and stimulation directions of the electrodes in the multi-group electrode array. The mapping table may be created by using genetic algorithm, ant algorithm, or other optimization algorithms.

The target stimulation directions of the virtual channels can be changed by selecting among different electrode carrying surfaces. For instance, when the plane electrode carrying surface is selected, the vertical range of the stimulation directions may be controlled; when the angular electrode carrying surface is selected, a stimulating range wider than that available in the case of a plane electrode carrying surface may be obtained; and when the spherical electrode carrying surface is selected, radial target stimulation directions may be obtained to have the widest stimulating range as compared to the plane and the angular electrode carrying surface. Therefore, the selection of the electrode carrying surface on the implant body may be determined according to the desired target stimulation directions of the virtual channels.

Therefore, when the electrical stimulation system using multi-group electrode array according to the present invention is applied in different neurostimulation technological fields, such as cochlear implant or deep brain stimulation, the virtual channels may be generated between corresponding electrodes located in the same electrode group or in two adjacent electrode groups. And, in the case of generating the virtual channel between two electrode groups, the positions of the virtual channels may be adjusted through proper planning of electrode groups and selection of corresponding electrodes. Moreover, the positions of corresponding electrodes for generating the virtual channels may also be adjusted by controlling different parameters, such as current intensity and current ratio. Further, the target stimulation sites and the stimulation directions of the virtual channels may be finely adjusted through selection of the positions of the electrode groups and the corresponding electrodes for generating the virtual channels, so as to achieve a high stimulation resolution.

The present invention has been described with some preferred embodiments thereof and it is understood that many changes and modifications in the described embodiments can be carried out without departing from the scope and the spirit of the invention that is intended to be limited only by the appended claims.

What is claimed is:

1. An electrical stimulation system using multi-group electrode array, comprising:
an implant body having an electrode carrying surface;
an electrode array provided on the electrode carrying surface of the implant body; the electrode array comprising a plurality of electrode groups, each of the electrode groups including a plurality of electrodes, and each of the electrodes having an independent power supply control; and
an electrode controller for receiving a control signal to drive a set of at least two corresponding electrodes, and powers supplied to the set of corresponding electrodes being regulated by the corresponding independent power supply controls thereof to thereby generate a virtual channel between the set of corresponding electrodes through interaction of powers supplied to the set of corresponding electrodes.

2. The electrical stimulation system using multi-group electrode array as claimed in claim 1, wherein each of the set of at least two electrodes is selected from the group consisting of the electrodes of the same electrode group and the electrodes of two adjacent electrode groups.

3. The electrical stimulation system using multi-group electrode array as claimed in claim 1, wherein the control signal comprises a control parameter, and the control parameter is an output ratio of powers.

4. The electrical stimulation system using multi-group electrode array as claimed in claim 3, further comprising a mapping table, from which a corresponding relationship between the control parameter and the virtual channel is able to be found.

5. The electrical stimulation system using multi-group electrode array as claimed in claim 4, wherein the mapping table is created by using genetic algorithm, ant algorithm or other optimization algorithm.

6. The electrical stimulation system using multi-group electrode array as claimed in claim 1, wherein the electrode carrying surface is selected from the group consisting of a plane surface, an angular surface, and a spherical surface.

7. The electrical stimulation system using multi-group electrode array as claimed in claim 1, wherein the electrodes in each of the electrode groups forming the electrode array are arranged into a pattern selected from the group consisting of a triangular pattern, a rectangular pattern, a pentagonal pattern, a hexagonal pattern, and a partial circular pattern.

8. The electrical stimulation system using multi-group electrode array as claimed in claim 1, wherein the electrodes are selected from the group consisting of a monopolar electrode, a bipolar electrode, and a multi-polar electrode.

9. The electrical stimulation system using multi-group electrode array as claimed in claim 1, wherein the powers supplied to the set of corresponding electrodes is selected from the group consisting of a voltage source and a current source.

10. The electrical stimulation system using multi-group electrode array as claimed in claim 1, wherein the electrical stimulation system is applicable to cochlear implant (CI) or deep brain stimulation (DBS).

11. An electrical stimulation method using multi-group electrode array, comprising the following steps:
providing an implant body having an electrode carrying surface;
providing an electrode array on the electrode carrying surface of the implant body; the electrode array comprising a plurality of electrode groups, each of the electrode groups including a plurality of electrodes; and the electrodes each having an independent power supply control;
receiving a control signal via an electrode controller to drive a set of at least two corresponding electrodes; and
regulating powers supplied to the set of corresponding electrodes by the corresponding independent power supply controls thereof, so as to generate a virtual channel between the set of corresponding electrodes through interaction of powers supplied to the set of corresponding electrodes.

12. The electrical stimulation method using multi-group electrode array as claimed in claim 11, wherein each of the set of at least two corresponding electrodes is selected from the group consisting of the electrodes of the same one electrode group and the electrodes of two adjacent electrode groups.

13. The electrical stimulation method using multi-group electrode array as claimed in claim 11, wherein the control signal comprises a control parameter, and the control parameter is an output ratio of powers.

14. The electrical stimulation method using multi-group electrode array as claimed in claim 13, further comprising a step of using a mapping table is used to look up a corresponding relationship between the control parameter and the virtual channel.

15. The electrical stimulation method using multi-group electrode array as claimed in claim 14, wherein the mapping table is created by using genetic algorithm, ant algorithm or other optimization algorithm.

16. The electrical stimulation method using multi-group electrode array as claimed in claim 11, wherein the electrode carrying surface is selected from the group consisting of a plane surface, an angular surface, and a spherical surface.

17. The electrical stimulation method using multi-group electrode array as claimed in claim 11, wherein the electrodes in each of the electrode groups forming the electrode array are arranged into a pattern selected from the group consisting of a triangular pattern, a rectangular pattern, a pentagonal pattern, a hexagonal pattern, and a partial circular pattern.

18. The electrical stimulation method using multi-group electrode array as claimed in claim 11, wherein the electrodes are selected from the group consisting of a monopolar electrode, a bipolar electrode, and a multi-polar electrode.

19. The electrical stimulation method using multi-group electrode array as claimed in claim 11, wherein the powers supplied to the set of corresponding electrodes is selected from the group consisting of a voltage source and a current source.

20. The electrical stimulation method using multi-group electrode array as claimed in claim 11, wherein the electrical stimulation system is applicable to cochlear implant (CI) or deep brain stimulation (DBS).
